(19) United States
(12) Patent Application Publication
(10) Pub. No.: US 2010/0096286 A1
(43) Pub. Date: Apr. 22, 2010

(54) Case for first aid supplies

(76) Inventors: Man For Ma, New Territories (CN); Daniel Peter Sterling, Norwalk, CT (US); Lee Webb, Fairfield, CT (US); Jonathan A. Lee, Milford, CT (US)

Correspondence Address: ALIX YALE & RISTAS LLP, 750 MAIN STREET, SUITE 1400, HARTFORD, CT, 06103, US

(21) Appl. No.: 12/288,050

(22) Filed: Oct. 16, 2008

(51) Int. Cl. A61B 19/02; B65D 1/24; B65D 25/28

(52) U.S. Cl. 206/363; 206/438; 220/500; 220/377; 220/752

(57) ABSTRACT

A case for first aid supplies has a housing is enclosed by a lid and is adapted to be both portable and mountable to a wall. The lid is an integral one piece member which includes a latch mechanism as well as an engagement strip for joining with the housing. An integral living hinge allows the lid to pivot. A stop mechanism limits the pivoting of the lid so that a tray may be formed.

### BACKGROUND

This disclosure relates generally to containers for first aid supplies. More particularly, this disclosure relates generally to containers which are structured to provide efficient organization of first aid supplies and allow for ready portability and efficient access to the supplies.

### SUMMARY

Briefly stated, a case for first aid supplies comprises a housing with a frontal lid. The housing has a support base and a pair of opposed contoured sides. The top has a central recess with a handle bridging between opposed sides of the recess and also has a transverse latch opening downwardly adjacent the central portion of the recess. A catch projects downwardly at a frontal portion of the latch opening. The housing has a plurality of compartments which open forwardly and a rear wall with an exterior surface. The frontal lid has a mounting strip fixedly mounted to a lower portion of the housing. The lid has an integral hinge which permits the lid to be pivoted from an open position allowing for access to the compartments to a closed position closing the compartments. The lid has an integral living reverse hinge-like latch which is receivable in the latch opening. The latch is engageable with the housing catch to latch the lid in the closed position. The latch has a projecting release tab which is depressable to release the latch. The lid has an integral tab which interferes with the structure projecting from the housing to retain the lid in a generally forward position projecting generally perpendicular to the upright housing to form a tray.

The lid is preferably transparent or semi-transparent. The housing base has three feet. The latch has an integral catch which engages the housing catch. The handle preferably has an arcuate shape. The projecting structure comprises a pair of side bars and a cross-bar which form an opening. The integral tab is received in the opening and bears against the cross-bar to limit the pivoting of the lid. The housing also has a pair of key-hole shaped openings at a rear portion.

### BRIEF DESCRIPTION OF THE DRAWINGS

FIG. 1 is a frontal perspective view of a case for first aid supplies;

FIG. 2 is an exploded perspective view, portions in phantom, of the case of FIG. 1;

FIG. 3 is a rear perspective view of the case of FIG. 1;

FIG. 4 is a bottom plan view of the case of FIG. 1;

FIG. 5 is a frontal perspective view of the case of FIG. 1 with the front lid in an open position;

FIG. 6 is a top plan view, partly broken away and partly in section, of the case of FIG. 1;

FIG. 7 is a side elevational view, partly broken away and partly in section, of the case of FIG. 1;

FIG. 8 is an interior fragmentary top plan view, partly in phantom, taken at the lower interior of the case and further illustrating the lid/housing inter-engagement;

FIG. 9 is a side sectional view, partly in diagram form, illustrating a pivoting stop mechanism for the case of FIG. 1; and

FIG. 10 is a frontal interior view of the pivot stop mechanism of FIG. 9.

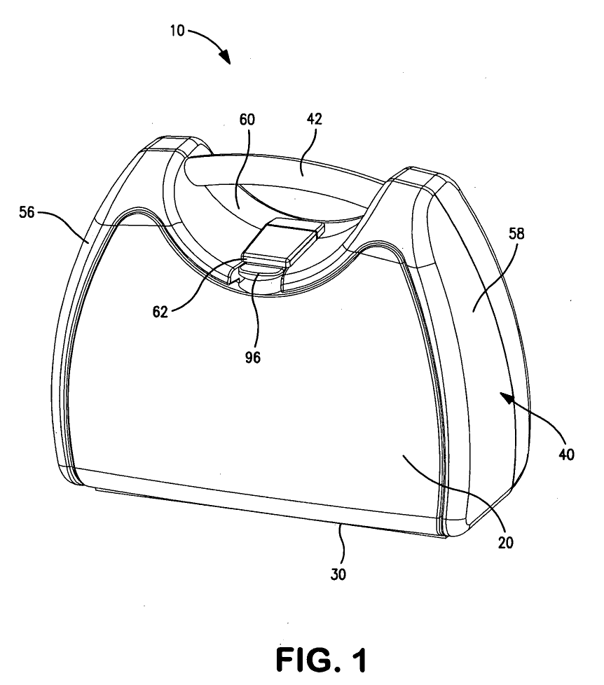

### DETAILED DESCRIPTION

With reference to the drawings wherein like numerals represent like components throughout the Figures, a case for storing first aid supplies is generally designated by the numeral 10. The case 10 is adapted to be easily portable and also is configured to be mounted to a wall or to sit upright on a support surface. The case 10 has an efficient contoured structure with a transparent or a semi-transparent frontal lid 20 to permit ready observation and identification of the contents within the housing. The case has a lightweight, rugged construction which withstands impact and is adaptable for multiple modes of passive and active usage, including an upright storage orientation on a support surface, mounting to a vertical wall and transport of the case to a location for usage.

The case comprises a housing module 40 which is configured for storing and organizing the first aid supplies and a frontal lid 20 which provides access to the supplies and also allows for a visual inspection of the supplies. A central arcuate handle 42 at upper opposed side portions of the housing module facilitates portability of the case. The housing module may generally be described as having a pleasing sculptured form which is especially suitable to function as a carrying case, but also is capable of being disposed in an upright orientation on a support surface and/or being mounted to a vertical wall.

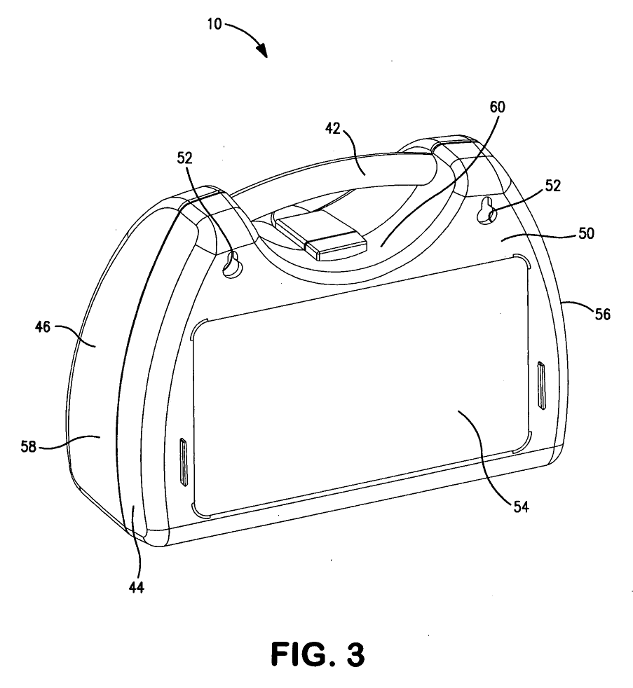

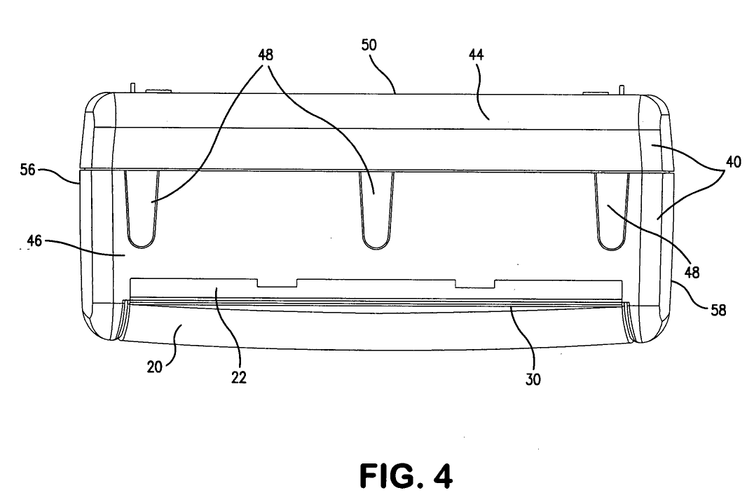

The housing module which, in one preferred embodiment, is constructed from a pair of molded components 44, 46 includes a support base which has three feet 48 to provide a stable upright support for the case. The case further has a generally planar rear panel 50, which includes at opposed spaced upper locations, a keyhole slot 52. The slots 52 facilitate removably mounting of the case to a pair of aligned fastener heads (not illustrated) projecting from a vertical wall. With reference to FIG. 3, the rear of the case also is preferably affixed with a chart 54 (not illustrated in detail) showing the various elements of the kit.

The opposed sides 56, 58 of the housing module have a tapered sculptured shape which upwardly taper toward the upper portions and form aesthetically pleasing contours. In one embodiment, the module is constructed from molded rear and frontal components 44, 46 which engage along a planar interface and are secured together by screws (not illustrated). The screw heads are accessible through the slots 52 at the rear of the case.

The module has a central arcuately contoured recess 60 at its upper portion. The handle 42 traverses the recess 60 from one side to another and the opposing ends of the handle are retained within corresponding openings through the sides of the recess. The handle shape and recess cooperate to provide ample room to comfortably grip the handle. A central portion of the recess is interrupted by a receptacle 62 which defines a transverse opening 64 through the front of the module. The upper wall of the opening is interrupted by a downwardly projecting catch 66 for a latch mechanism, as will be further described.

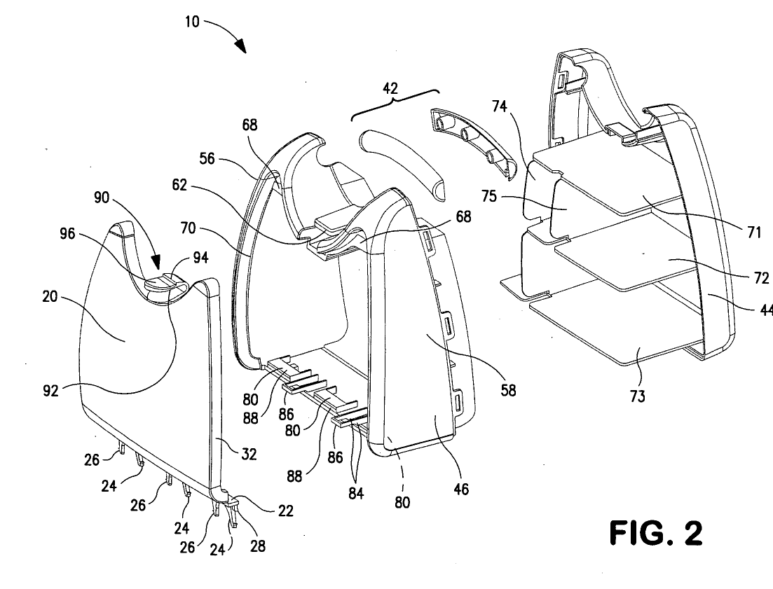

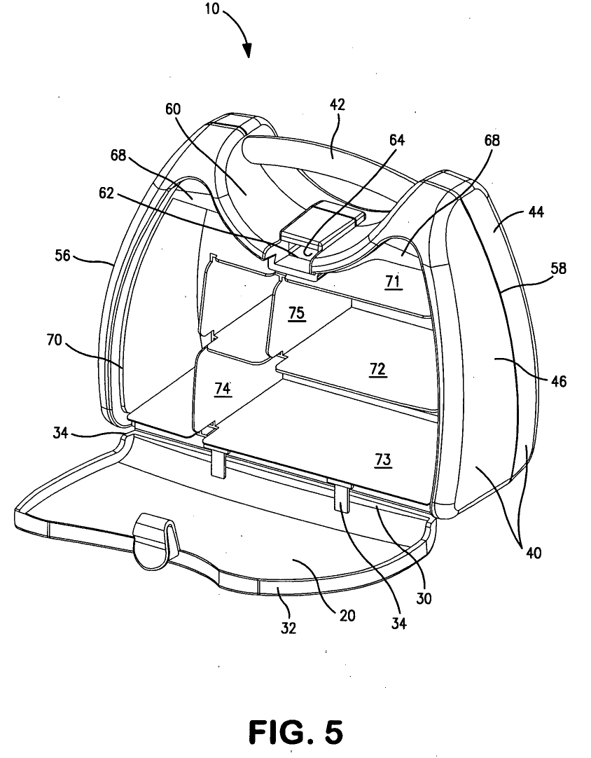

With reference to FIGS. 2 and 5, the forward portion of the housing module forms a frontal opening which is closed by the lid. The upper portions include a recessed abutment 68 at opposed locations which form a stop for the lid 20. An interior semi-arcuate recessed rim 70 at opposed interior sides of the module also function as a stop for the lid. The recessed abutments 68 and rims 70 are co-planar.

The interior of the housing module has forwardly projecting panels 71-75 which intersect and form compartments for the first aid supplies. In the illustrated embodiment, essentially five compartments in cooperation with the various interior walls of the housing module are formed. Three generally horizontal panels 71-73 and two vertical panels 74-75 intersect to define the compartments. Preferably, the panels are integral molded structures which are formed with the housing module and extend from the rear component 44. Numerous alternative panel/compartment configurations or no such compartments are possible.

The inside lower base of the module has three spaced integral brackets 80 which include a platform and define a retainer slot 88, as will be further described. Disposed between the brackets 80 is a pair of integral projections 84 which each project forwardly from the lower frontal opening and include a cross-member 86 which forms a catch.

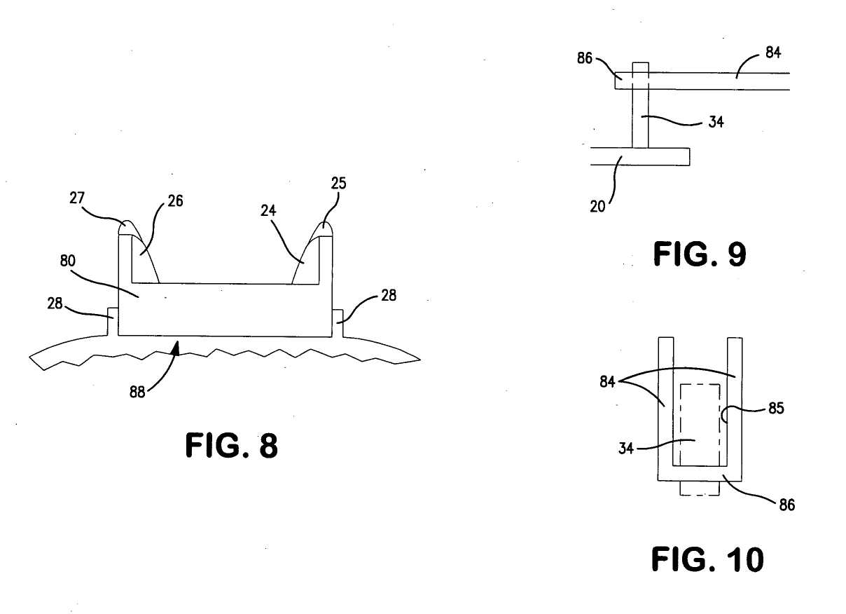

With reference to FIGS. 2 and 8, the lid 20 is preferably a transparent or semi-transparent molded member which includes an integral mounting strip 22 with a series of opposed engagement tabs 24, 26. A locating boss 28 is spaced from each of the tabs 24, 26. The tabs project rearwardly wherein each tab engages sides of a bracket and inwardly extends through the retainer slot and engages the inside of the bracket. Each tab 24, 26 includes a corresponding rearward catch 25, 27 which engages the rear surface of the platform defining the slot. The rear edge of the mounting strip is forced rearwardly through the brackets so that the ramps clear the rear edge of the brackets and abut the edges thereof in a snap-fit-type connection to retain the lid with the lower portion of the housing module. The locating bosses 28 engage the outer sides of the brackets 80. The mounting strip 22 and thus the lid 20 is rigidly affixed to the housing module 40.

Generally forwardly from the mounting strip, a living hinge 30 is formed. The lid includes an inwardly protruding rim. 32 which is generally complementary to that of the recessed engagement rim 70 and the stops 68 of the housing module. The lid 20 has a concave upper face which projects forwardly so that the upper portion of the lid has a shape which inversely mirrors the general shape of the recess 60.

The lid can then be pivoted about the hinge 30 so that the rim 32 engages the stop 68 of the housing module and encloses the module. The lid 20 is preferably a molded member formed from transparent or semi-transparent material which has a rigid construction. With reference to FIGS. 5, 9 and 10, the lower inside portion of the lid includes a pair of projecting pivot stops 34. As the cover is pivoted to the closed position, the stops 34 are pivotally received in openings 85 of the fixed catches 86. When the lid is pivoted fully to the forward open position, each fixed catch 86 is engaged by a pivot stop 34 to essentially limit the pivoting motion so that the lid is in a substantially perpendicular relationship to the frontal opening of the case. The structure at the inside of the lid, including the peripheral rim 32, essentially functions as a tray for the supplies when the case is mounted to a vertical wall.

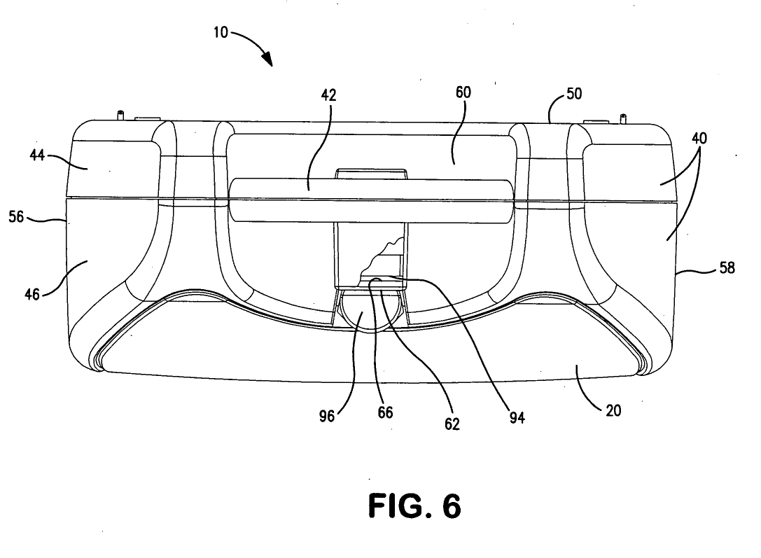

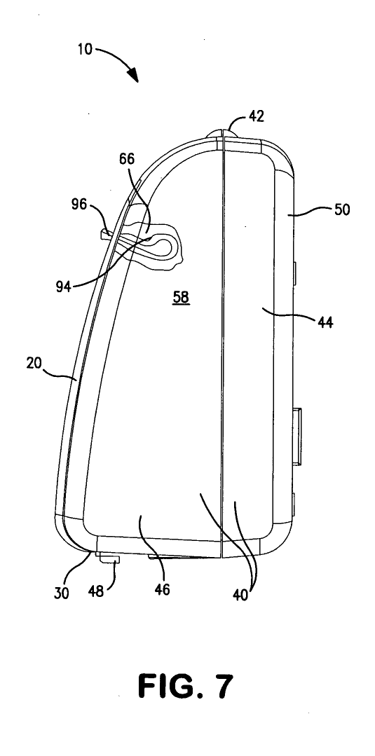

With additional reference to FIGS. 6 and 7, the upper central portion of the cover has an integral reverse U-shaped latch 90. The upper leg 92 of the latch is resiliently upwardly biased and is configured to define a reverse catch 94. A release tab 96 extends forwardly from the catch. As the cover is pivoted to the closed position, the hinge-like latch is forced rearwardly into the catch receptacle. The latch 90 which carries the catch 94 is sufficiently resilient that the catch upwardly biases against the top of the receptacle until the catch 94 clears the forward catch 66 at the underside of the receptacle. The resilient ramp-like structure and path of the latch urges the two catches 66 and 94 to engage to essentially lock the cover in the closed position.

The lid 20 may be efficiently opened by downwardly depressing the frontal tab 96 to clear the engagement of the catches and by forwardly pivoting the cover to open same. The opening movement will be limited by the pivot stops 34 which engage the fixed catches 86 at the lower portion of the housing module. It will be appreciated that the foregoing case 10 can be constructed in an efficient manner with few parts and the hinge-like lid 20 can be efficiently secured to the lid for pivotal motion for opening and closing same. In addition, the latching mechanism at the top is efficiently manufactured as an integral part of the lid to provide a reliable latch 90 which can be easily released for gaining access to the components.

While the foregoing has been set forth for the purpose of describing preferred embodiments, the foregoing description should not be deemed a limitation of the invention herein. Accordingly, various modifications, adaptations and alternatives may occur to one skilled in the art without departing from the spirit and the scope of the present invention.

1. A case for first aid supplies comprising:
a housing having a support base and a pair of opposed contoured sides and a top defining a central recess with a handle bridging between opposed sides of said recess and defining a transverse latch opening downwardly adjacent a central portion of said recess with a catch projecting downwardly at a frontal portion of said latch opening, said housing having a plurality of compartments which open forwardly and a rear wall with an exterior surface; and
a frontal lid having a mounting strip fixedly mounted to a lower portion of said housing and defining an integral hinge which permits said lid to be pivoted from an open position which allows for access to said compartments to a closed position enclosing said compartments, said housing having an integral living reverse hinge-like latch receivable in said opening and engageable with said catch to latch said lid in the closed position, said latch having a projecting release tab depressable to release said latch and said lid having an integral tab which interferes with a structure projecting from said housing to retain said lid in a generally forward position projecting generally forwardly to form a tray.

2. The case of claim 1 wherein said lid is transparent or semi-transparent.

3. The case of claim 1 wherein said base has three feet.

4. The case of claim 1 wherein said latch has an integral catch which engages said housing catch.

5. The case of claim 1 wherein said handle has an arcuate shape.

6. The case of claim 1 wherein said projecting structure comprises a pair of side bars and a crossbar which define an opening.

7. The case of claim 6 wherein said integral tab is received in said opening and bears against said crossbar to limit the pivoting of the lid.

8. The case of claim 1 wherein said rear wall defines a pair of keyhole shaped openings at a rear portion thereof.

9. The case of claim 1 wherein a contents list is affixed to said exterior surface.

10. A case for first aid supplies comprising:
a housing having a support base and a pair of opposed contoured sides and a top defining a central recess with a handle bridging between opposed sides of said recess and defining a transverse latch opening downwardly adjacent a central portion of said recess with a catch projecting downwardly at a frontal portion of said latch opening, said housing having a rear wall with an exterior surface and forming an enclosure; and
a frontal lid having a mounting strip fixedly mounted to a lower portion of said housing and defining an integral hinge which permits said lid to be pivoted from an open position which allows for access to said enclosure to a closed position enclosing said enclosure, said housing having an integral living reverse hinge-like latch receivable in said opening and engageable with said catch to latch said lid in the closed position, said latch having a projecting release tab depressable to release said latch and said lid having an integral tab which interferes with a structure projecting from said housing to retain said lid in a generally forward position projecting generally forwardly to form a tray.

11. The case of claim 10 wherein said lid is transparent or semi-transparent.

12. The case of claim 10 wherein said base has three feet.

13. The case of claim 10 wherein said latch has an integral catch which engages said housing catch.

14. The case of claim 10 wherein said handle has an arcuate shape.

15. The case of claim 10 wherein said projecting structure comprises a pair of side bars and a crossbar which define an opening.

16. The case of claim 15 wherein said integral tab is received in said opening and bears against said crossbar to limit the pivoting of the lid.

17. The case of claim 10 wherein said rear wall defines a pair of keyhole shaped openings at a rear portion thereof.

18. The case of claim 10 wherein a contents list is affixed to said exterior surface.
